(19) United States
(12) Patent Application Publication
(10) Pub. No.: US 2024/0146810 A1
(43) Pub. Date: May 2, 2024

(54) EDGE COMPUTING SYSTEM FOR LOCALLY PROCESSING DATA IN A CLINICAL NETWORK

(71) Applicant: FRESENIUS VIAL SAS, Brézins (FR)

(72) Inventor: Christophe Reynier, Chirens (FR)

(21) Appl. No.: 18/558,520

(22) PCT Filed: May 3, 2022
(86) PCT No.: PCT/EP2022/061774

§ 371 Date: Nov. 1, 2023

(30) Foreign Application Priority Data
May 21, 2021 (EP) 21315086.5

(51) Int. Cl. H04L 67/12; G16H 40/67; H04L 43/04

(52) U.S. Cl. CPC H04L 67/12; G16H 40/67; H04L 43/04

(57) ABSTRACT

An edge computing system for locally processing data in a clinical network administered by a remote central sewer device (100), comprising: at least one medical care device (SAA, SAN), at least one first edge-node (3A), and a monitoring device (7) assigned to the at least one medical care device (SAA, SAN); the at least one first edge-node (3A) comprising: a connection interface (13) adapted to establish a data connection with the at least one medical care device (SAA, SAN), and with at least one of the remote central sewer device (100) and the monitoring device (7); and a processing module (15) adapted to: (i) aggregate raw data from the at least one medical care device (SAA, SAN), (ii) combine at least metadata with the aggregated raw data to generate processed data, and (iii) transmit the processed data to the monitoring device (7) and/or to the remote central sewer device (100) via the data connection. Also described is a method for locally processing data in a clinical environment, and a computer program product.

The invention relates to an edge computing system for locally processing data in a clinical network administered by a remote central server device according to claim 1, and to a method for locally processing data in a clinical environment.

Current state of the art systems for processing data in clinical environments are divided into two network areas, a so-called point of care network and a hospital IT network.

The point of care network provides automatic electronic data capture of patient-related and healthcare information, as well as of device operational data. A medical/health device communication standard is specified in ISO/IEEE 11073. The Hospital IT network facilitates exchange and storage of healthcare information and can be specified by standards, such as DICOM, HL7 v2, or HL7 FHIR.

Hence, the point of care network, as currently known in the art, only focuses on aspects of obtaining healthcare information in the proximity of the patient bed and room elements. The processing of the obtained information, such as specific data processing and visualization, however, needs a higher perspective and data consolidation. Such specific data processing relies in the prior art commonly on a central server architecture administering the hospital IT network.

Such a central server requires hardware capabilities which are hard to predict in order to process the vast amount of possible requests originating from the high number of distant devices. Also, such a central server architecture presents a potential single point of failure. Hence, if the central server fails, the entire system may stop working as desired and intended. A further problem arises if the communication with the central server is impaired.

It is an object of the instant invention to provide a computing system and a method for processing data in a more effective and reliable approach.

This object is achieved by an edge computing system for locally processing data in a clinical network administered by a remote central server device, comprising the features of claim 1.

Accordingly, the edge computing system comprises:
at least one medical care device, at least one first edge-node, and a monitoring device assigned to the at least one medical care device; the at least one first edge-node comprising:
   a connection interface adapted to establish a data connection with the at least one medical care device, and with at least one of the remote central server device and the monitoring device; and
   a processing module adapted to:
   (i) aggregate raw data from the at least one medical care device,
   (ii) combine at least metadata with the aggregated raw data to generate processed data, and
   (iii) transmit the processed data to the monitoring device and/or to the remote central server device via the data connection.

The edge computing system is administered by the remote central server device, which essentially must not be part of the actual edge computing system. In an example, however, the edge computing system can comprise the remote central server device.

The at least one medical care device can be understood as a passive or active device that is associated with a patient and that can be located in the vicinity of the patient. For example, the medical care device can be located at the patient's bedside. Examples for passive medical care devices are devices for monitoring at least one vital function of the patient such as the blood pressure, heartbeat, etc. and to which it can be therefore also referred to as “patient monitoring devices”. An example, for an active medical care device can be an infusion device, such as an infusion pump.

The monitoring device can be an electronic physiological monitoring device that may also use electronic medical charting and recording. Typically, one such monitoring device can be used to monitor the status of a number of medical care devices at the same time. For example, the monitoring device can comprise a computer, for example a desktop computer, and a display, for example a touch screen display. The computer can run a software such as a software that provides infusion status to effectively organise and help users, such as nurses, to prioritise their actions. In an example, 24 rooms/48 beds can be monitored. However, in further examples, more or less rooms/beds can be monitored.

The at least one first edge-node can be herein understood as a computing device that can act as a portal for allowing communication between medical care devices connected to the edge-node, to allow communication with other edge-nodes as well as with possible other medical care devices connected to the other edge-nodes, the monitoring device, and the remote central sever device. Also, in correspondence with the name “edge-node”, the computing device can be, in general, seen as running at least one functionality at the edge of a physical location. For example, the first edge-node can communicate directly with the remote central server device, or communicate indirectly with the remote central server device, e.g. via at least one second edge node to which a number of first edge nodes may be connected to.

The first edge-node comprises a connection interface adapted to establish a data connection with the at least one medical care device, and with at least one of the remote central server device and the monitoring device. The term “interface” can be used to refer to a hardware interface and/or a software interface that allow the first edge-node to establish the data connection. The data connection can be established permanently or temporarily, i.e. when needed, and which can be an uplink and/or downlink data connection. Also, the data connection with at least one of the remote central server device and the monitoring device can be established directly with at least one these devices or indirectly, e.g. via a second edge-node.

The raw data that the processing module is adapted to aggregate from the at least one medical care device can be understood as data that is sent or read by the medical care device. Each medical care device can have a specific payload and protocol to transfer/receive data. Here, the term “payload” can be used to refer to the “actual data” being sent or read. The term “protocol” can be used to refer to a software protocol that makes use of its own software, or to an open-source protocol that makes use of an open-source software.

The metadata that the processing module is adapted to combine with the aggregated raw data to generate processed data can comprise a location identifier, a patient identifier, a prescription identifier, etc. In this context, the term “metadata” can be generally understood as data that provides information about other data and can be used in examples to refer to specific locations in the topology of a hospital. The specific locations can be the patient-, room-, ward-, and/or organisation level. Also, a mere combination of metadata and aggregated raw data can be referred to as “rich data”.

The processing module is also adapted to transmit the processed data to the monitoring device and/or to the remote central server device via the data connection.

Advantageously, the edge computing system allows to segment and partition the network and the data management. Each edge-node can host and run different functional data processing which enables in each topology level specific data processing, visualization or notification in correspondence with the actual need of each topology level.

Hence, the edge computing system described herein allows to more efficiently process and forward data in a distributed approach.

In an example, the connection interface is further adapted to retrieve analytical data from the remote central server device and/or from the monitoring device, and the processing module is further adapted to combine the analytical data with the raw data and the metadata into the processed data.

The term “analytical data” can be used herein to refer to data that has been obtained previously from aggregated raw data and metadata, and/or which has been extracted from larger amounts of data such as from previously aggregated raw data and metadata using algorithms to obtain meaningful information. In a more specific example, where an infusion is to be administered, the term “analytical data” can be data in regard to the remaining infusion time, i.e. the time that elapses until a target volume is reached. The accordingly generated smart data can be visualised as progress, for example by means of displaying a gauge, on the display of the monitoring device.

Advantageously, smart data can be generated when analytical data is combined with the raw data and the metadata into the processed data. Smart data can be used both to gain new insights using the aggregated raw data and metadata and to create models that can be used to analyse data.

In an example, the edge computing system comprises a computing resource, preferably a software library, wherein the connection interface is further adapted to establish a data connection with the computing resource, and to retrieve analytical data from the computing resource, and the processing module is further adapted to combine the analytical data with the raw data and the metadata into the processed data.

Advantageously, the analytical data can be stored in a computing resource such as, for example, a drug library which comprises data pertaining to a patient's past and current medication, and which can be accessed when needed to obtain analytical data for generating smart data.

In an example, the edge computing system comprises a further first edge-node, wherein the connection interface of the first edge-node is further adapted to establish a data connection with the further first edge-node, wherein the further first edge-node is in communication with at least one further medical care device, and wherein the processing module of the first edge-node is further adapted to transmit or receive the processed data to or from the further first edge-node.

Here, the further first edge-node can be of the same type than the first edge-node as described above. Also, the medical care device and the further medical care device can be of the same type.

Advantageously, the first edge-node and the further first edge-node can be in direct communication with each other, to exchange data, i.e. without the need to route communication through a central server.

In an example, the computing system comprises at least a second edge-node, comprising:
a connection interface adapted to establish a data connection with the at least one first edge-node, and with at least one of the remote central server device and the monitoring device, and
a processing module adapted to receive and/or transmit the processed data from the at least one first edge-node.

Advantageously, a plurality of first edge-nodes can be connected to the second-edge node. Also, the data connection with at least one of the remote central server device and the monitoring device can be made directly, or indirectly, e.g. via higher layer edge-nodes.

In an example, the connection interface of the second edge-node is further adapted to:
receive analytical data from the at least one first edge-node, and from at least one of the remote central server device and the monitoring device; and
the processing module is further adapted to:
combine the analytical data into the processed data, and
transmit the processed data to the monitoring device and/or to the remote central server device via the data connection.

The analytical data can be, for example, received from the remote central server device, the first edge-node, the further first edge-node, or the computing resource.

Advantageously, the second edge-node can generate smart data, which can be then transmitted to the monitoring device and/or to the remote central server.

In an example, the first edge-node is assigned to a patient, and the second edge-node is assigned to a hospital patient-room, or the first edge-node is assigned to a hospital patient-room, and the second edge-node is assigned to a hospital ward.

In an example, at least one third edge-node, preferably a plurality of further higher level edge-nodes are arranged between the second edge-node and the central server device.

The edge computing system can, for example, comprise the at least one third edge-node, and further higher level edge-nodes.

In an example, the at least one third edge-node is assigned to a hospital building and/or to a hospital.

In an example, the at least one medical care device is adapted to disconnect the data connection with the first edge-node when moving out of a range of the first edge-node and to establish a data connection with the further first edge-node when moving into a range of the further first edge-node.

For example, the at least one medical care device can be operable with a number, or with all of the first edge-nodes used in the system, such as for example being operable at least with the first edge-node and the further first edge-node.

Advantageously, the medical care device can automatically disconnect and re-connect to first edge-nodes when it is moved from one patient-room to another patient-room.

In an example, the at least one medical care device is at least one of an infusion device and a patient monitoring device.

Here, the infusion device is can be a syringe pump and/or a volumetric pump. The patient monitoring device can be an EEG monitoring device, an exhalation monitoring device, and/or a device monitoring blood glucose, blood pressure, heart rate, and/or oxygen concentration.

In an example, the connection module of the at least one first edge-node is adapted to connect to the at least one medical care device via a wireless connection, preferably a Wireless LAN connection.

Advantageously, an existing network can be utilized for connecting the first edge-node to the medical care device.

In an example, the connection module of the at least one first edge-node and/or of the at least one second edge-node is adapted to be operable with a communications network according to EN ISO 11073, and/or according to a Fast Healthcare Interoperability Resources, FHIR, standard, and/or a Health Level Seven, HL7, standard.

Advantageously, the first and second edge-nodes can be able to connect to the point of care network and to the hospital IT network.

The invention also relates to an edge computing system for locally processing data in a clinical network, comprising:
a plurality of first edge-nodes, wherein each first edge-node is connectable to at least one medical care device to exchange data with the at least one medical care device, and wherein the first edge-nodes are operable to exchange data among the plurality of first edge-nodes; and
at least one second edge-node, wherein the second edge-node is connectable to each first edge-node, and to at least one of a third edge-node, and a remote central server device to exchange data.

The invention also relates to a method for locally processing data in a clinical environment, using an edge computer system comprising at least one first edge-node, at least one medical care device, and a monitoring device assigned to the at least one medical care device, wherein the method comprises the steps of:
establishing, by a connection interface of the at least one first edge-node, a data connection with the at least one medical care device, and with at least one of a remote central server device and the monitoring device; and
performing, by a processing module of the at least one first edge-node, the steps of:
(i) aggregating raw data from the at least one medical care device,
(ii) combining at least metadata with the aggregated raw data to generate processed data, and
(iii) transmitting the processed data to the monitoring device and/or to the remote central server device via the data connection.

In an example, the method comprises:
combining the analytical data with the raw data and the metadata into the processed data; and/or
sending/receiving processed data to/from at least one further first edge-node.

In another example, the method comprises:
sending/receiving processed data to/from at least one second edge-node, and
establishing at the second edge-node a data connection with at least one of a further first edge-node, the remote central server device and the monitoring device.

Further, the invention relates to computer program product comprising a computer readable storage medium having program instructions stored thereon, the program instructions executable by a processor to perform a method as described herein.

The idea underlying the invention shall subsequently be described in more detail by referring to the embodiments shown in the figures. Herein:

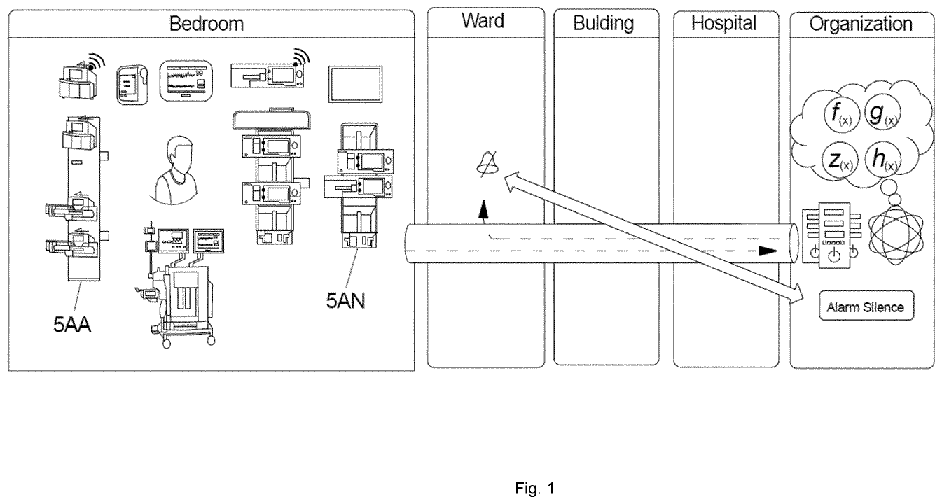

FIG. 1 shows a schematic drawing of centralized data processing in a clinical environment as known in the prior art;

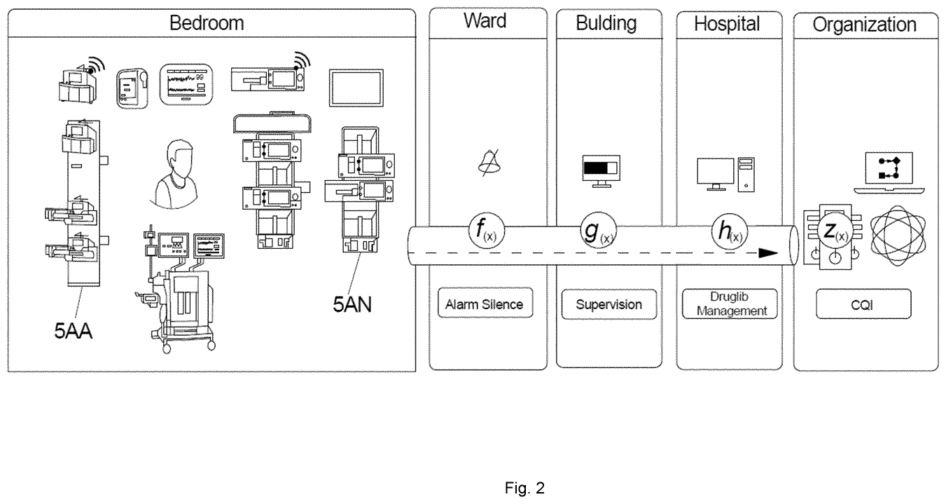

FIG. 2 shows a schematic drawing of de-centralized data processing in a clinical environment employing the edge computing system for locally processing data;

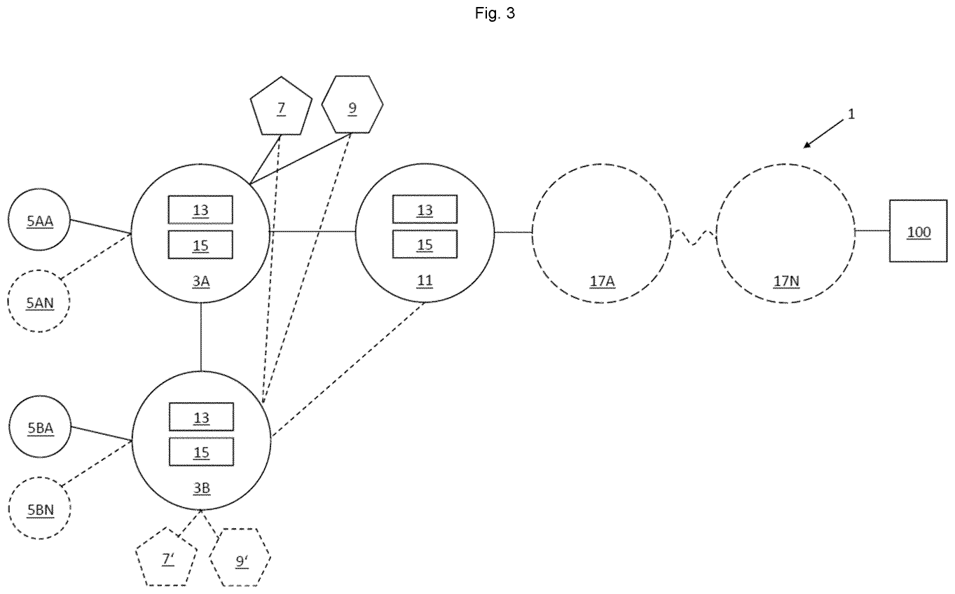

FIG. 3 shows a schematic drawing of the edge computing system for locally processing data according to an embodiment of the invention;

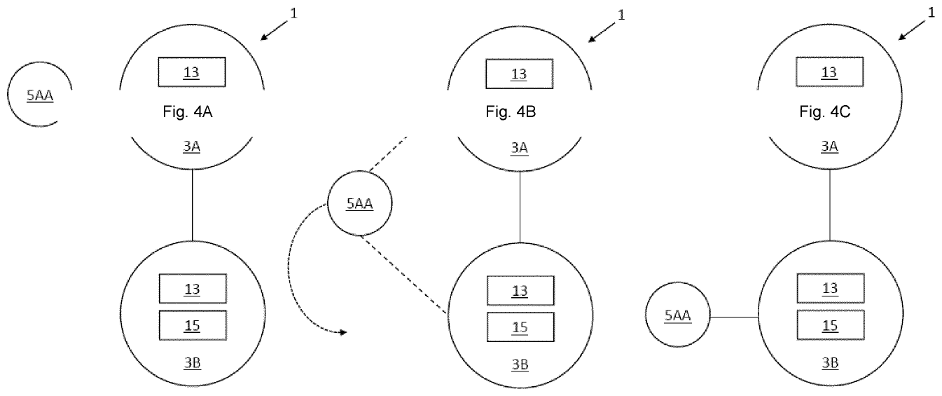

FIGS. 4A-4C show schematic drawings of a medical care device disconnecting from the first edge-node and connecting to the further first edge-node;

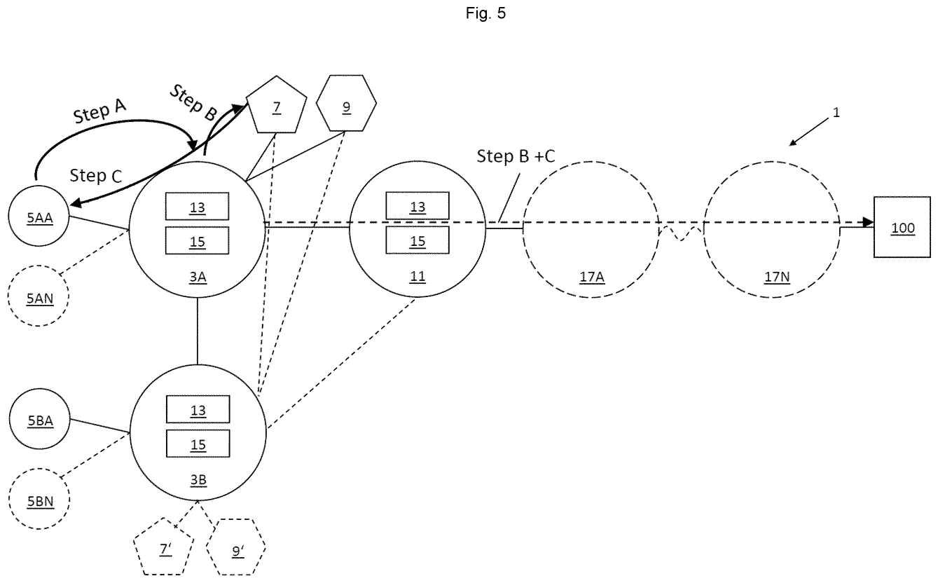

FIG. 5 shows schematic steps of a case study employing the edge computing system;

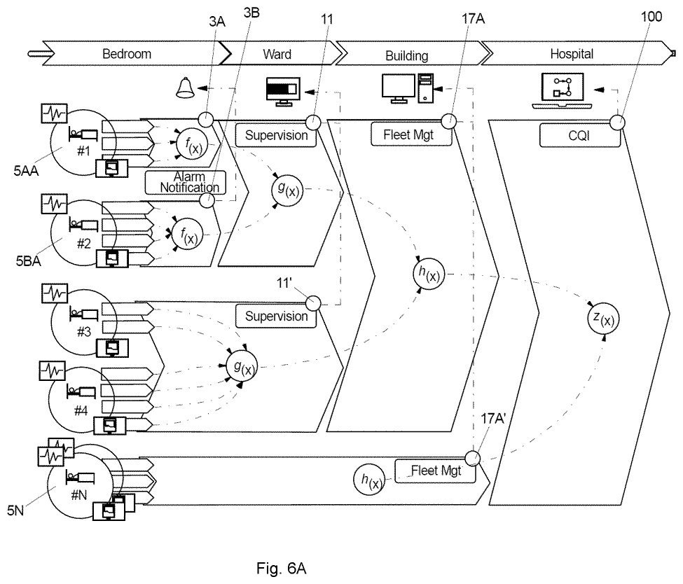

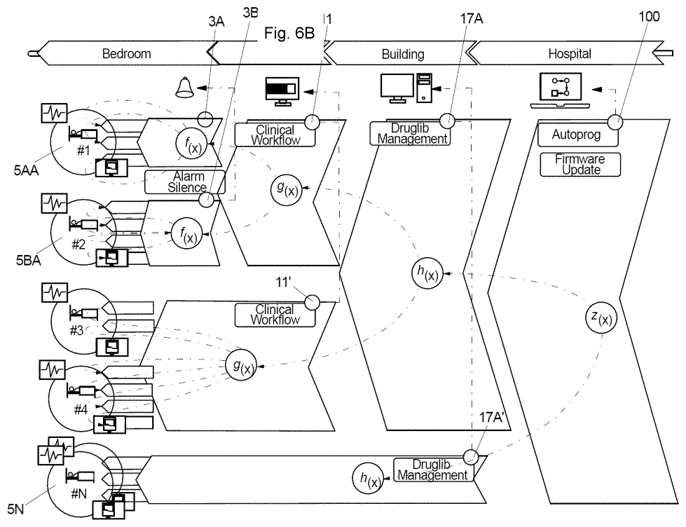

FIGS. 6A, 6B show schematic upstream and downstream data flows in de-centralized data processing in a clinical environment employing the edge computing system;

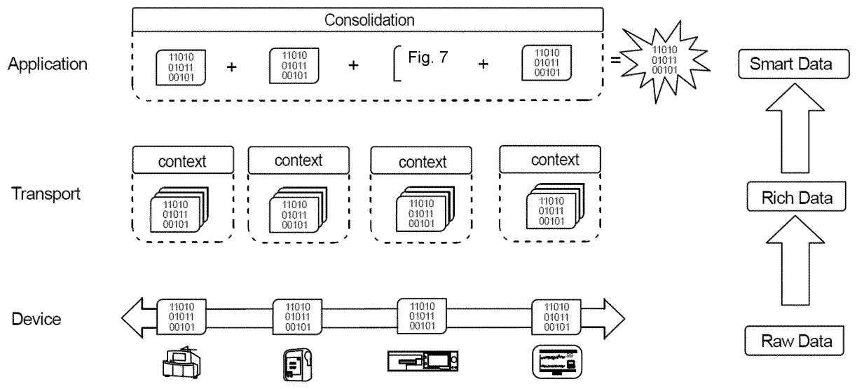

FIG. 7 shows a simplified layer model comprising data types according to embodiments of the invention; and

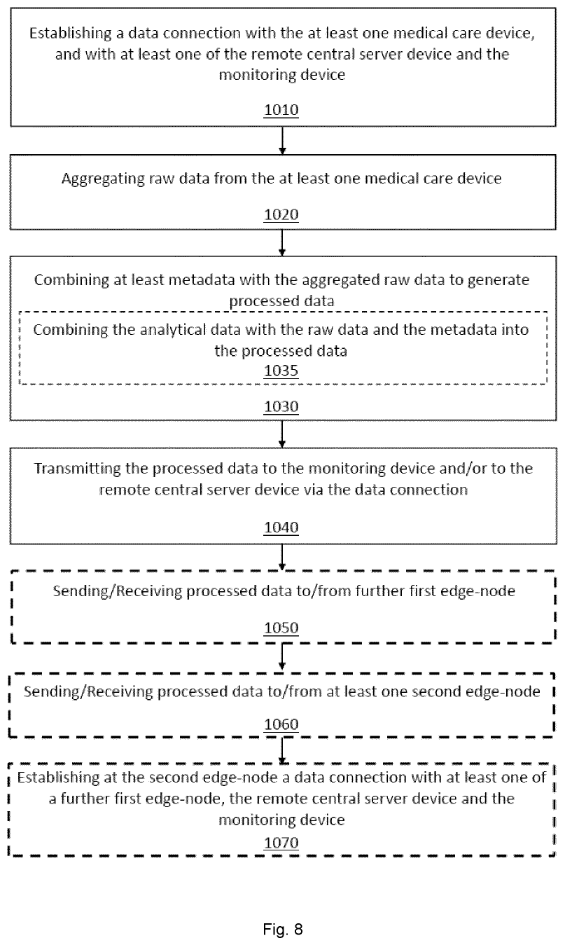

FIG. 8 shows a method flow according to an embodiment of the invention.

FIG. 1 shows a schematic drawing of centralized data processing in a clinical environment as it is known in the prior art. The setup shown in FIG. 1 is a simplified representation of data processing at the different levels of the clinical environment. On the very left side of FIG. 1, a patient's bed room is shown and a plurality of medical care devices 5AA-5AN that can be located in the patient's bed room, and that are generally located at the patient's bedside, such as devices for monitoring the patient's vital functions, and/or infusion pumps. As shown in FIG. 1, some of the medical care devices 5AA-5AN comprise a WiFi interface for wireless communication.

The medical care devices are normally monitored with a monitoring device at the ward level. However, as shown by the horizontal arrows in dashed lines, data from the medical care devices is first routed via the building and hospital level to a central server located at the organization level. In the prior art example of FIG. 1 an alarm situation is shown for explanatory purposes. Here, the detection of the alarm and then silencing the alarm takes place via the central server.

Utilizing centralized data processing as shown in FIG. 1, requires hardware capabilities which are hard to predict beforehand in order to process the vast amount of possible requests originating from the high number of possible devices. Also, such a central server architecture presents a potential single point of failure. Hence, if the central server fails, or if the communication with the server is interrupted, the entire system may stop working as desired and intended.

FIG. 2 shows a schematic drawing of de-centralized data processing in a clinical environment employing an edge computing system for locally processing data according to an embodiment of the invention. The different levels of the clinical environment shown in FIG. 2 correspond to the levels shown in FIG. 1.

At the different levels of the clinical environment different operations or functions f(x), g(x), h(x), z(x) are exemplarily shown in FIG. 2. The operations f(x), g(x), h(x), z(x) are handled directly by each edge-node at the designated level (not shown in FIG. 2).

Here, an edge-node can be in charge of the f(x) functions, for example silencing an alarm, at the ward level, while a further edge-node at a higher level is in charge of g(x) functions, such as for example for displaying the conditions of several beds or wards and supervising them, at the building level. An edge-node higher up than the previous edge-node is in charge, e.g. is connected to a drug library at the hospital level, and can retrieve data from/add data to the drug library. In the example shown in FIG. 2, the central server is at the organization level, centralizing the information and performing advanced functions z(x) like continuous quality improvement (CQI).

The de-centralized data processing is described in greater detail with reference to the following figures.

FIG. 3 shows a schematic drawing of the edge computing system 1 for locally processing data according to an embodiment. The edge computing system 1 that is shown in FIG. 3 can be employed to the clinical environment using de-centralized data processing as shown before in FIG. 2.

The edge computing system 1 as shown in FIG. 3 comprises two first edge-nodes 3A, 3B. However, in a minimum configuration the edge computing system 1 can also comprise just one first edge-node 3A in embodiments of the invention. Also, shown is at least one medical care device 5AA, 5BA that is connected to a respective first edge-node 3A, 3B. However, more than one medical care device can be connected to each first edge-node 3A, 3B, which is indicated by medical care devices 5AN, 5BN that are depicted using dashed lines.

The first edge-nodes 3A, 3B also comprise a connection interface 13 adapted to establish a data connection with the medical care devices 5AA, 5AN, 5BA, 5BN and with at least one of the remote central server device 100 and the monitoring device 7 shown in FIG. 3. Here, the data connection can be made directly via the corresponding device, or via other edge-nodes. For example, the data connection from the first edge-node 3A to the central server 100 is made via a second edge-node 11 as described in more detail below. Also, as shown in FIG. 3, the data connection between the further first edge-node 3B and the monitoring device 7 can be made via the first edge-node 3A, but could be also established directly as indicated by the dashed line connecting the monitoring device 7 with the further first edge-node 3B. Further, FIG. 3 shows a computing resource 9 which comprises a software library to which the first edge-node 3A is connected to. The further first edge-node 3B and the computing resource 9 can be connected via the first edge-node 3A, but could be also connected directly as indicated by the dashed line connecting the computing resource 9 to the further first edge-node 3B. Also, in embodiments of the invention, a further monitoring device 7′ and a further computing resource 9′ can be additionally or alternatively connected to the further first edge-node 3B.

The processing module 15 of the first edge-nodes 3A, 3B is in the shown embodiment adapted to:
(i) aggregate raw data from at least one medical care device 5AA, 5AN, 5BA, 5BN,
(ii) combine at least metadata with the aggregated raw data to generate processed data, and
(iii) transmit the processed data to the monitoring device 7 and/or to the remote central server device 100 via the data connection.

In the shown embodiment, the connection interface 13 is further adapted to retrieve analytical data from the remote central server device 100 and/or from the monitoring device 7, and the processing module 15 is further adapted to combine the analytical data with the raw data and the metadata into the processed data.

The different data types are explained in more details with reference to FIG. 7.

Also, shown in FIG. 3 is the connection of the first edge-node 3A and the further first edge-node 3B, and that the further first edge-node 3B is in communication with at least one further medical care device 5BA. The processing module 15 of the first edge-node 3A is further adapted to transmit or receive the processed data to or from the further first edge-node 3B.

Here, the shown further first edge-node 3B is of the same type than the first edge-node 3A. Accordingly, the first edge-node 3A and the further first edge-node 3B can be in direct communicate with each other, to exchange data, i.e. without the need to route communication through the central server device 100. Also, shown in FIG. 3 is a dashed line between the further first edge-node 3B and the second edge-node 11 to indicate that the further first edge-node 3B could be also in direct communication with the second edge-node 11.

Also, the second edge-node 11, which is shown in FIG. 3, comprises a connection interface 13 and a processing module 15. Here, the same reference numbers are used than for the corresponding components of the first edge-nodes 3A, 3B since these components can be essentially similar. The connection interface 13 of the second edge-node 11 is adapted to establish a data connection with the at least one first edge-node 3A, and with at least one of the remote central server device 100 and the monitoring device 7. The connection interface 13 of the second edge-node 11 is further adapted to receive analytical data.

The monitoring device 7 and the further first edge-node 3B can be connected directly to the second edge-node 11, or the connection can be made via the first edge-node 3A as shown in FIG. 3.

The processing module 15 of the second edge-node 11 is adapted to receive and/or transmit the processed data from the at least one first edge-node 3A. Also, the processing module 15 is further adapted to combine the analytical data into the processed data, and to transmit the processed data to the monitoring device 7 and/or to the remote central server device 100 via the data connection.

The depicted first edge-nodes 3A, 3B can be assigned to respective hospital patient-rooms, and the second edge-node 11 can be assigned to a hospital ward. In further embodiments, at least a third edge-node 17A, or a plurality of further higher level edge-nodes 17N can be arranged between the second edge-node 11 and the central server device 100. In FIG. 3 the third edge-node 17A and the higher level edge-node 17N are both shown by dashed lines to indicate that these entities are merely optional.

FIGS. 4A-4C show schematic drawings of a medical care device 5AA disconnecting from the first edge-node 3A and connecting to the further first edge-node 3B.

In FIGS. 4A-4C the first edge-node 3A and the further first edge-node 3B which are previously shown in FIG. 3 are depicted. For the sake of simplicity, just one medical care device 5AA is shown, but it will be apparent that further medical care devices could be connected to first edge-node 3A and to the further first edge-node 3B when the medical care device 5AA is moved from the first edge-node 3A to the second edge-node 3B as shown in FIGS. 4A-4C.

In FIG. 4A the medical care device 5AA is shown connected to the first edge-node 3A, while in FIG. 4B the dashed lines and the dashed arrow indicate that the medical care device 5AA is moved out of a range of the first edge-node 3A and into the range of the further first edge-node 3B. Here, the medical care device 5AA can maintain the data connection with the first edge-node 3A, while establishing a data connection with the further first edge-node 3B when moving into the range of the further first edge-node 3B. The data connection with the first edge-node 3A is disconnected once the connection with the further first edge-node 3B is established.

If the first edge-node 3A and the further first edge node 3B are located at a greater distance from each other, the medical care device 5AA can also completely disconnect from the first edge node 3A when being moved out of the range of the first edge-node 3A, and later connect to the further first edge node 3B when the medical care device 5AA is moved into the range of the further first edge-node 3B.

In FIG. 4C, the medical care device 5AA is connected to the further first edge-node 3B, after it was moved from the first edge-node 3A to the further first edge-node 3B.

In the shown embodiment, the connection is a WiFi connection and the range is determined by the strength of a corresponding WiFi signal.

FIG. 5 shows schematic steps of a case study employing the edge computing system 1 shown in FIG. 3.

To highlight the inventive concept of the edge computing system 1 shown in FIG. 3, the following case study is presented in FIG. 5. In FIG. 5 the schematic drawing of the edge computing system 1 of FIG. 3 is shown and additional arrows are added to indicate the below explained steps of the case study:

Step A: In a first step, it is assumed that an alarm event is generated in a medical care device 5AA. For example, the medical device 5AA could comprise an infusion pump and the alarm event could indicate a malfunction of the infusion pump.

Data indicating the alarm event is then sent upstream from the medical care device 5AA to the first edge-node 3A.

Step B: The alarm event is then forwarded by the first edge-node 3A to monitoring device for notifying the user.

For forwarding the alarm to the monitoring device 7, raw data is first aggregated from the medical care device 5AA and comprises, in the present case, data in regard to an infusion process. The first-edge node 3A then combines the aggregated raw data with metadata, which comprise, in the present case, data in regard to an ID of the infusion pump, the patient, the patient room, etc. into processed data which is then transferred to the monitoring device 7.

Optionally, the processed data is also sent to the central server device 100 via the second edge-node 11, and via higher up edge-nodes 17A, 17N located upstream between the second edge-node 11 and the remote central server device 100 as indicated by the dashed arrow extending from the second edge-node 11 to the remote central server device 100, where the occurrence of the alarm event can be logged.

Step C: The alarm event is then acknowledged by the user on the monitoring device 7 and the acknowledgement is returned to the medical care device 5AA that initiated the alarm event via the first edge-node 3A, and optionally to the central server device 100.

The above case study highlights that each edge-node can host and run different functional data processing which enables in each topology level specific data processing, visualization or notification in correspondence with the actual need of the corresponding topology level. The functional data processing can be completely independent from the data processing that takes place in the central server device 100.

FIGS. 6A and 6B show the upstream and downstream data flow in de-centralized data processing in a clinical environment employing the edge computing system for locally processing data according to the embodiment of the invention as shown in FIGS. 3 to 5.

A first edge node 3A and a further first edge node 3B are exemplarily shown at the bedroom level, where also medical care devices 5AA and 5BA are located.

In FIG. 6A the upstream data flow is shown, where raw data originating from the medical care devices 5AA and 5BA is received at the corresponding first edge nodes 3A, 3B. In the present case, the raw data comprises data in regard to the administering of an infusion fluid, such as the infusion speed. The alarm event denoted as “alarm notification” indicates a malfunction of the infusion pump, which is represented by aggregated raw data. Metadata comprising a patient ID or an ID of the corresponding medical care device 5AA, 5BA is added to the raw data. Afterwards, the data is sent to the second edge-node 11 at the ward-level. Here, the g(x) functions, comprise functions in regard to supervision, such as monitoring the conditions of several beds. The dashed line arrows extending from the second edge node 11 higher up to a third edge-node 17A indicate the data which is sent upstream to the third edge-node 17A. Here, the h(x) functions, comprise functions in regard to fleet management, such as monitoring the conditions of all beds in a building.

The central server 100 is located at the organization level, centralizing the information and performing advanced functions z(x), such as continuous quality improvement (CQI). As it can be seen from FIG. 6A, data from the lowest level can be transferred to the central server 100, while data processing at lower levels of the edge computing system does not need to involve the central server 100 as it will be apparent by the downstream data flow shown in FIG. 6B.

In the downstream direction, the advanced functions z(x) at the organization level can comprise, as shown in FIG. 6B, functions providing updates, such as providing firmware updates, to entities at the lower levels. The functions h(x) at the building level can comprise providing data from a drug library, to the lower levels. Here, the g(x) functions at the ward level can comprise providing data in regard to the clinical workflow to the bedroom level. As already described with reference to FIG. 5, the downstream functions f(x) at the bedroom level can comprise returning an acknowledgement to the medical care device 5AA that initiated an alarm event.

At all levels shown in FIGS. 6A and 6B upstream and downstream data can be combined.

FIG. 7 shows a simplified layer model comprising data types according to embodiments of the invention.

At the bottom in FIG. 7 raw data is shown that can be understood as data that is sent or read by the medical care device. Each medical care device can have a specific payload and protocol to transfer/receive data.

The metadata that the processing module is adapted to combine with the raw data to generate processed data can comprise a location identifier, a patient identifier, a prescription identifier, etc. As already described herein, the metadata can refer to specific locations in the topology of a hospital. The specific locations can be the patient-, room-, ward-, and/or organisation level. Also, a mere combination of metadata and raw data can be also referred to as “rich data”.

Smart data is generated at the application layer when adding analytical data to the rich data. Analytical data can be data that was extracted from larger amounts of data such as from previously aggregated raw data and metadata using algorithms to obtain meaningful information. The accordingly generated smart data can be visualised as progress, for example by means of displaying a gauge, on the display of the monitoring device. Also, historical data, or data from further medical care devices can be used for generating corresponding smart data, such as for example for displaying a ratio of the fluid being administered by different medical care devices and/or comparing the ratio to thresholds.

FIG. 8 shows a method flow according to an embodiment of the invention. The method comprises the following steps:
Establishing 1010 a data connection with the at least one medical care device, and with at least one of the remote central server device and the monitoring device;
Aggregating 1020 raw data from the at least one medical care device;
Combining 1030 at least metadata with the aggregated raw data to generate processed data; and
Transmitting 1040 the processed data to the monitoring device and/or to the remote central server device via the data connection.

The method can also comprise further optional steps that are not essential for the invention, and which are therefore depicted in boxes delimitated by a dashed line:
Combining 1035 the analytical data with the raw data and the metadata into the processed data;
Sending/Receiving 1050 processed data to/from at least one further first edge-node;
Sending/Receiving 1060 processed data to/from at least one second edge-node; and
Establishing 1070 at the second edge-node a data connection with at least one of a further first edge-node, the remote central server device and the monitoring device.

### LIST OF REFERENCE NUMERALS

1 Edge Computing System
3A, 3B First Edge-Node
5AA, 5AN, 5BA, 5BN, 5N Medical Care Device
7, 7′ Monitoring Device
9, 9′ Computing Resource
11, 11′ Second Edge-Node
13 Connection Interface
15 Processing Module
17A-17N Further Edge-Node
100 Remote Central Server Device
1000 Method for Locally Processing Data in a Clinical Environment
1010 Establishing
1020 Aggregating
1030 Combining
1035 Combining Analytical Data
1040 Transmitting
1050 Sending/Receiving to/from Further First Edge-Node
1060 Sending/Receiving to/from Second Edge-Node
1070 Establishing at the Second Edge-Node a Data Connection
Step A Alarm Event is generated
Step B Alarm Event is forwarded
Step C Alarm Event is silenced
f(x), g(x), h(x), z(x) Functions in Clinical Environment

1. An edge computing system for locally processing data in a clinical network administered by a remote central server device, comprising:
at least one medical care device, at least one first edge-node, and a monitoring device assigned to the at least one medical care device;
the at least one first edge-node comprising:
a connection interface configured to establish a data connection with the at least one medical care device, and with at least one of the remote central server device and the monitoring device; and
a processing module configured to:
(i) aggregate raw data from the at least one medical care device,
(ii) combine at least metadata with the aggregated raw data to generate processed data, and
(iii) transmit the processed data to the monitoring device and/or to the remote central server device via the data connection.

2. The edge computing system of claim 1, wherein the connection interface is further configured to retrieve analytical data from the remote central server device and/or from the monitoring device, and the processing module is further configured to combine the analytical data with the aggregate raw data and the metadata into the processed data.

3. The edge computing system of claim 1, further comprising a computing resource wherein the connection interface is further configured to establish a data connection with the computing resource, and to retrieve analytical data from the computing resource, and the processing module is further configured to combine the analytical data with the aggregate raw data and the metadata into the processed data.

4. The edge computing system of claim 1, further comprising a further first edge-node, wherein the connection interface of the at least one first edge-node is further configured to establish a data connection with the further first edge-node, wherein the further first edge-node is in communication with at least one further medical care device, and wherein the processing module of the at least one first edge-node is further configured to transmit or receive the processed data to or from the further first edge-node.

5. The edge computing system of claim 1, further comprising at least a second edge-node, comprising:
a connection interface configured to establish a data connection with the at least one first edge-node, and with at least one of the remote central server device and the monitoring device, and
a processing module configured to receive and/or transmit the processed data from the at least one first edge-node.

6. The edge computing system of claim 5, wherein the connection interface of the second edge-node is further configured to:
receive analytical data from the at least one first edge-node, and from at least one of the remote central server device and the monitoring device; and
the processing module of the second edge-node is further configured to:
combine the analytical data from the at least one first edge-node and from the at least one of the remote central server device and the monitoring device into the processed data, and
transmit the processed data to the monitoring device and/or to the remote central server device via the data connection.

7. The edge computing system of claim 5, wherein the first edge-node is assigned to a patient, and the second edge-node is assigned to a hospital patient-room, or the first edge-node is assigned to a hospital patient-room, and the second edge-node is assigned to a hospital ward.

8. The edge computing system of claim 5, wherein at least one third edge-node is arranged between the second edge-node and the remote central server device.

9. The edge computing system of claim 8, wherein the at least one third edge-node is assigned to a hospital building and/or to a hospital.

10. The edge computing system of claim 4, wherein the at least one medical care device is configured to disconnect the data connection with the at least one first edge-node when moving out of a range of the at least one first edge-node and to establish a data connection with the further first edge-node when moving into a range of the further first edge-node.

11. The edge computing system of claim 1, wherein the at least one medical care device is at least one of an infusion device and a patient monitoring device.

12. The edge computing system of claim 1, wherein the connection interface of the at least one first edge-node is configured to connect to the at least one medical care device via a wireless connection.

13. The edge computing system of claim 1, wherein the connection interface of the at least one first edge-node and/or of the at least one second edge-node is adapted configured to be operable with a communications network according to EN ISO 11073, and/or according to a Fast Healthcare Interoperability Resources (FHIR) standard, and/or a Health Level Seven (HL7) standard.

14. A method for locally processing data in a clinical environment, using an edge computer system, comprising at least one first edge-node, at least one medical care device, and a monitoring device assigned to the at least one medical care device, wherein the method comprises the steps of:
establishing, by a connection interface of the at least one first edge-node, a data connection with the at least one medical care device, and with at least one of a remote central server device and the monitoring device; and
performing, by a processing module of the at least one first edge-node OM, the steps of:
(i) aggregating raw data from the at least one medical care device,
(ii) combining at least metadata with the aggregated raw data to generate processed data, and
(iii) transmitting the processed data to the monitoring device and/or to the remote central server device via the data connection.

15. The method of claim 14, further comprising:
combining the analytical data with the aggregated raw data and the metadata into the processed data; and/or
sending/receiving the processed data to/from at least one further first edge-node

16. The method of claim 14, further comprising:
sending/receiving the processed data to/from at least one second edge-node, and
establishing at the second edge-node a data connection with at least one of a further first edge-node, the remote central server device and the monitoring device.

17. A computer program product comprising a computer readable storage medium having program instructions stored thereon, the program instructions executable by a processor to perform a method according to claim 14.

18. The edge computing system of claim 3, wherein the computing resource comprises a software library.

19. The edge computing system of claim 8, wherein the at least one third edge-node comprises a plurality of further higher level edge nodes.

20. The edge computing system of claim 12, wherein the wireless connection is a Wireless LAN connection.
